(19) United States
(12) Patent Application Publication
(10) Pub. No.: US 2019/0002815 A1
(43) Pub. Date: Jan. 3, 2019

(54) Large-scale Bioreactor

(71) Applicant: 3D Biotek, LLC, Bridgewater, NJ (US)

(72) Inventors: Zongsen Wang, Princeton, NJ (US); Wing Lau, Basking Ridge, NJ (US); Faribourz Payvandi, Belle Mead, NJ (US); Peter Materna, Metuchen, NJ (US)

(73) Assignee: 3D Biotek, LLC, Bridgewater, NJ (US)

(21) Appl. No.: 16/128,041

(22) Filed: Sep. 11, 2018

Related U.S. Application Data
(63) Continuation-in-part of application No. 15/686,211, filed on Aug. 25, 2017.
(60) Provisional application No. 62/556,646, filed on Sep. 11, 2017.
(60) Provisional application No. 62/636,039, filed on Feb. 27, 2018.
(60) Provisional application No. 62/380,414, filed on Aug. 27, 2016.

(51) Int. Cl. C12M 1/12; C12M 1/00; C12M 1/26; C12M 1/42; C12M 1/34

(52) U.S. Cl. CPC C12M 25/14; C12M 29/00; C12M 23/34; C12M 33/08; C12M 41/00; C12M 35/04; C12M 41/26; C12M 41/28; C12M 33/00

(57) ABSTRACT

In an embodiment of the invention, there may be provided a bioreactor having tissue scaffolds and having culture medium perfused therethrough. There may be multiple independent culture chambers and reservoirs or sub-reservoirs. Sensors can provide for individually controlling conditions in various culture chambers, and various culture chambers can be operated differently or for different durations. It is possible to infer the number of cells or the progress toward confluence from the fluid resistance of the scaffold, based on flowrate and pressure drop. Harvesting may include any combination or sequence of; exposure to harvesting reagent; vibration; liquid flow that is steady, pulsatile or oscillating; passage of gas-liquid interface through the scaffold. Vibration and flow can be applied so as to reinforce each other.

### CROSS-REFERENCE TO RELATED DOCUMENTS

This patent application claims the benefit of provisional U.S. patent application Ser. No. 62/556,646 filed Sep. 11, 2017; and provisional U.S. patent application Ser. No. 62/636,039, filed Feb. 27, 2018. This patent application is a continuation-in-part of nonprovisional U.S. patent application Ser. No. 15/686,211, filed Aug. 25, 2017 and published as US20180057784, which claims the benefit of provisional U.S. patent application Ser. No. 62/380,414, filed Aug. 27, 2016. All of these are incorporated herein by reference in their entireties.

### FIELD OF THE INVENTION

Embodiments of the invention pertain to bioreactors.

### BACKGROUND OF THE INVENTION

Bioreactors are used to expand a population of cells, such as stem cells or other anchorage dependent cells. However, improvements are still desirable, such as in regard to ease of use, automation, reproducibility of procedures, and the number of cells that can be produced. It is desirable to culture cells so as to produce as many as billions of cells or even more than ten billion cells from a given culturing process using a given apparatus. Also, in connection with such a process, it is desirable to provide a system such that if contamination were to occur somewhere in the system, it does not necessarily result in loss of an entire batch.

### SUMMARY OF THE INVENTION

In an embodiment of the invention, there may be provided a bioreactor system for culturing cells, the bioreactor system comprising spatially fixed scaffolds upon which the cells can grow, the bioreactor system having a liquid supply system for perfusing liquid through the scaffolds, wherein the bioreactor system comprises a plurality of culture chambers each containing some of the scaffolds, the culture chambers having respective flow paths therethrough for flow of the liquid, wherein the bioreactor system comprises a plurality of reservoirs or a plurality of sub-reservoirs, wherein the bioreactor system has a control device to direct, to various of the plurality of culture chambers at a given time, respective flows of the liquid that are different from flows to others of the culture chambers with respect to flowrate of the liquid or flow direction of the liquid or duration of flow of the liquid.

An embodiment of the invention comprises a method for retrieving cells from a bioreactor system, the method comprising: providing a bioreactor system comprising a spatially fixed scaffold upon which the cells can grow, the bioreactor system having a liquid supply system for perfusing a liquid through the scaffolds, wherein the bioreactor system comprises a culture chamber containing some of the scaffolds, the culture chamber having a flow path therethrough for flow of said liquid; culturing cells in the bioreactor on the scaffold; and performing, in any combination and in any sequence, any one or more of: (a) exposing said cells to a harvesting reagent; (b) applying vibration to said bioreactor system; (c) applying oscillatory flow of liquid through said scaffold; (d) applying pulsatile flow of liquid through said scaffold; or (e) causing a liquid-gas interface to pass through said scaffold.

An embodiment of the invention comprises a method of culturing cells, the method comprising: providing a bioreactor system comprising a spatially fixed scaffold upon which the cells can grow, the bioreactor system having a liquid supply system for perfusing a liquid through the scaffolds, the liquid supply system comprising a pump, wherein the liquid supply system comprises a pressure measuring device for measuring a pressure generated by the pump or a means for measuring electrical power consumed in operating the pump; culturing cells on the scaffolds; optionally harvesting the cells that have been cultured; and during either the culturing or the harvesting or both, determining a flow resistance of the scaffold using information about flowrate of the liquid in combination with either information about the pressure measured by the pressure measuring device or information about the electrical power consumption of said pump.

### BRIEF DESCRIPTION OF THE ILLUSTRATIONS

Embodiments of the invention are further described but are in no way limited by the following illustrations.

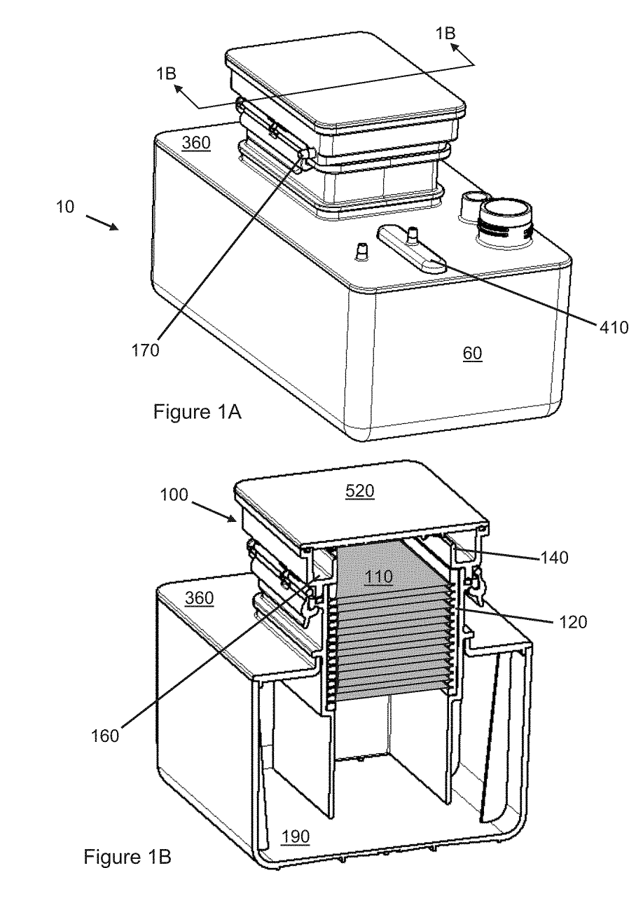

FIG. 1A is a three-dimensional perspective view of a culture chamber mounted above a reservoir.

FIG. 1B is a sectional view of FIG. 1A.

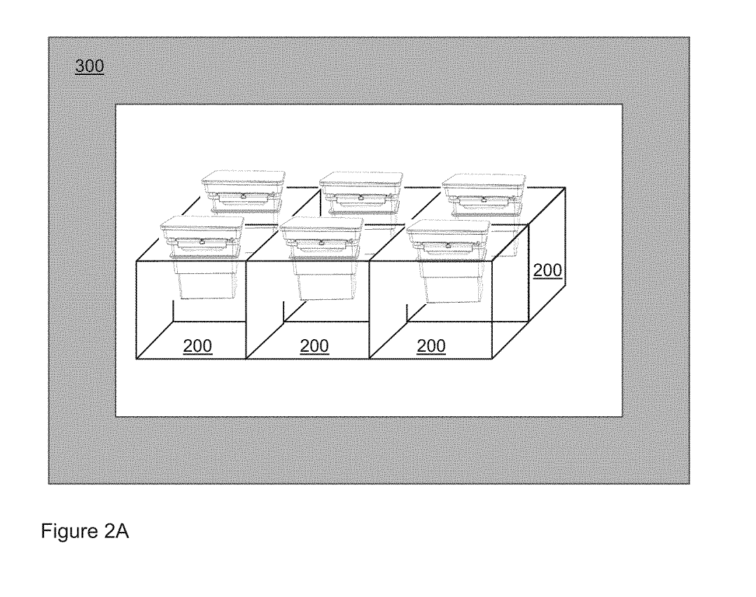

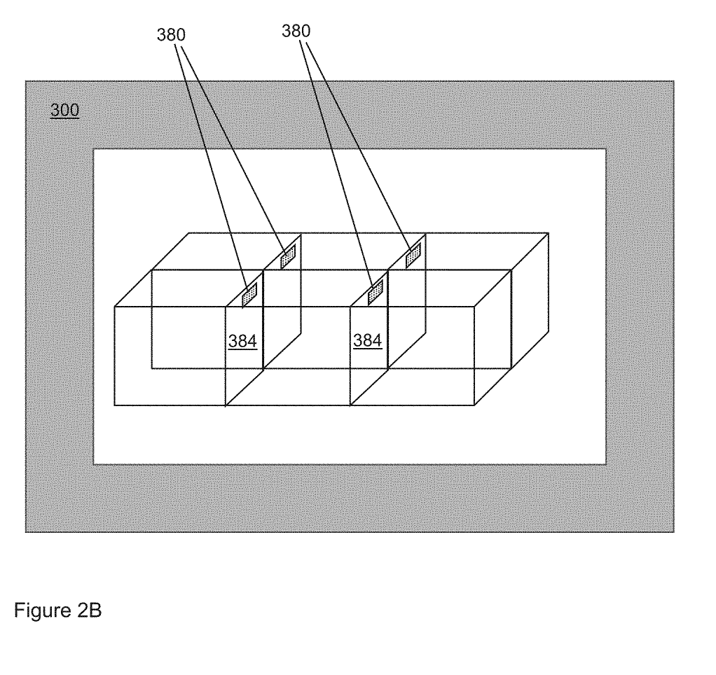

FIG. 2A is a three-dimensional perspective view of a reservoir assembly having six sub-reservoirs, with a culture chamber mounted above each sub-reservoir, all of which is enclosed by an incubator.

FIG. 2B is similar to FIG. 2A, except that the culture chambers are omitted for clarity, and further showing side-flow filters mounted in walls that separate adjacent sub-reservoirs.

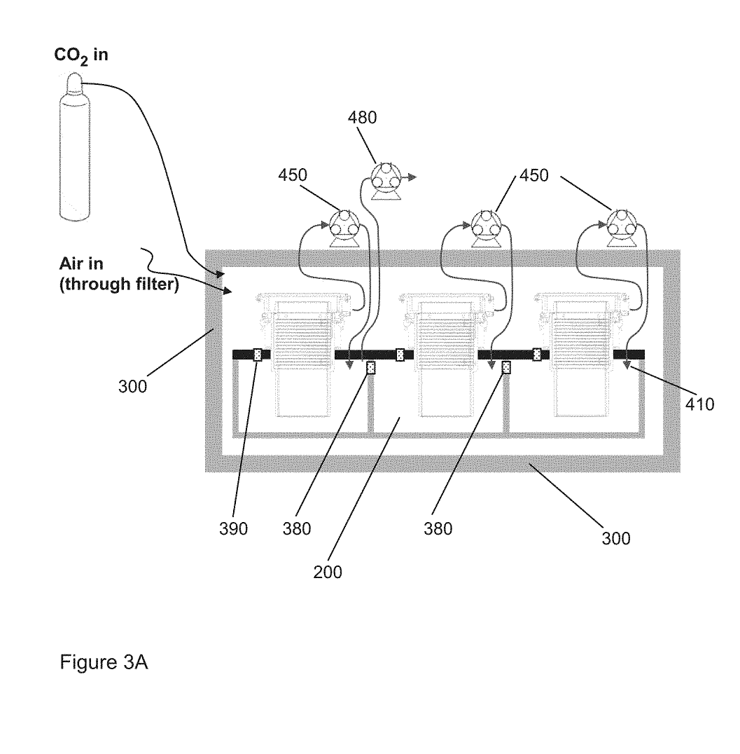

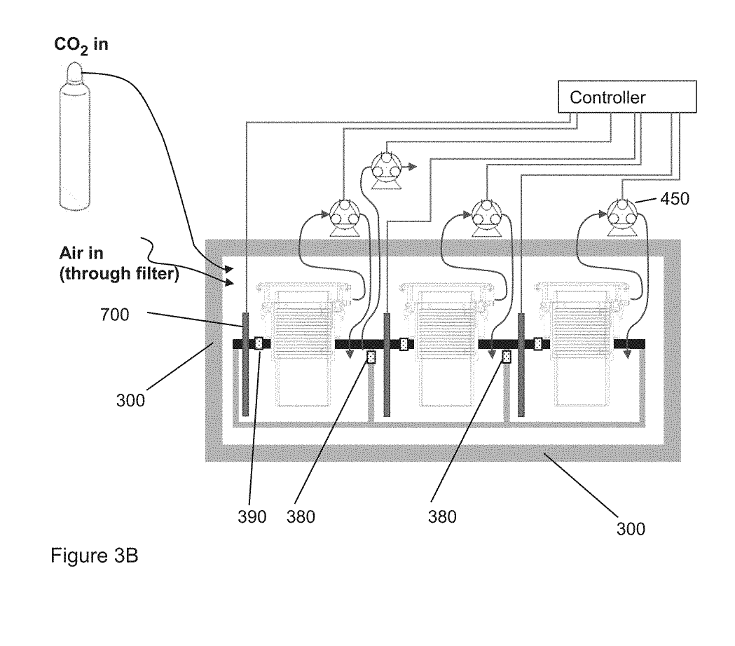

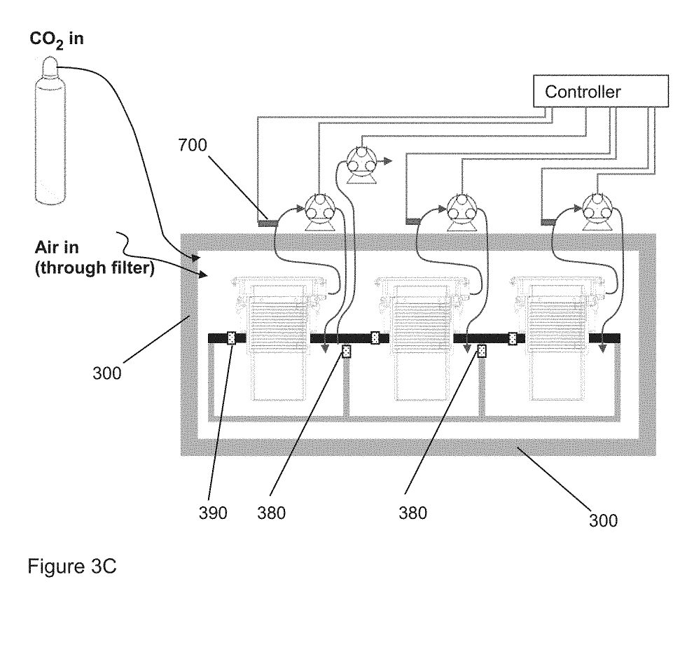

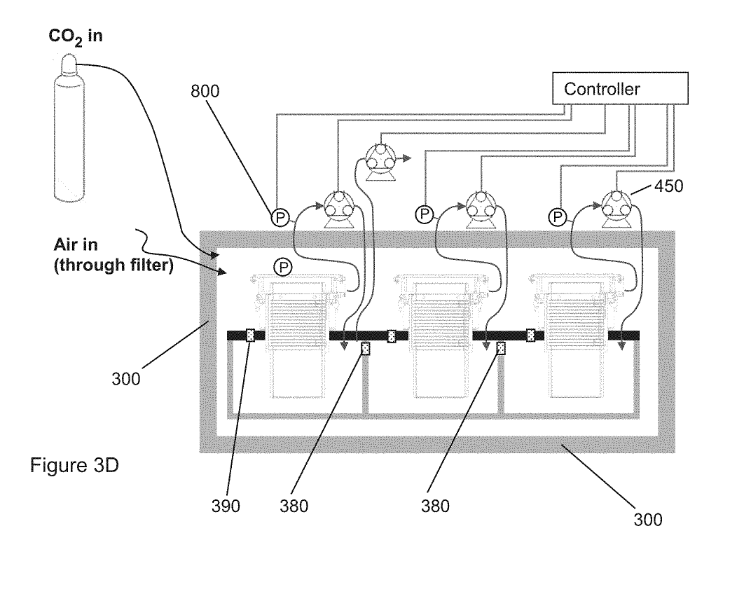

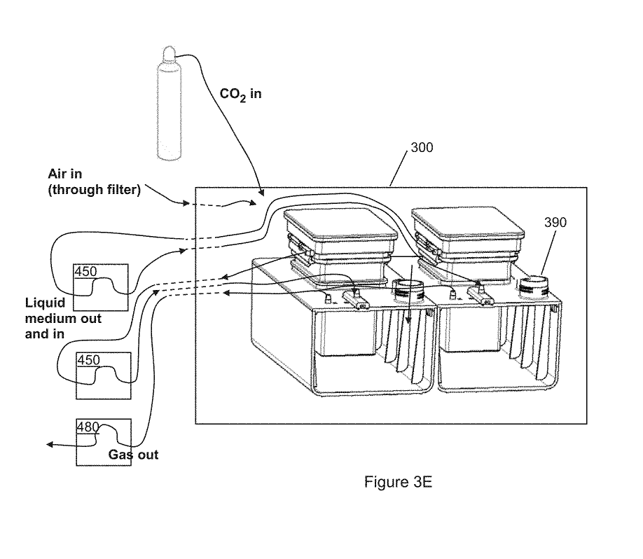

FIG. 3A is a side view showing three sub-reservoirs, with a culture chamber in each sub-reservoir, and further showing flowpaths for liquid and for gas. Each sub-chamber has its own liquid pump.

FIG. 3B is similar to FIG. 3A but additionally showing a control system that controls operation of the pump for each sub-reservoir according to a parameter sensed by an immersed sensor.

FIG. 3C is similar to FIG. 3B except that the sensor is in contact with fluid in tubing that is external to the sub-reservoir.

FIG. 3D is similar to FIG. 3C except that the sensor is a pressure transducer.

FIG. 3E is a cutaway view showing two completely independent reservoirs inside an incubator, with a culture chamber in each sub-reservoir, and further showing flowpaths for liquid and for gas.

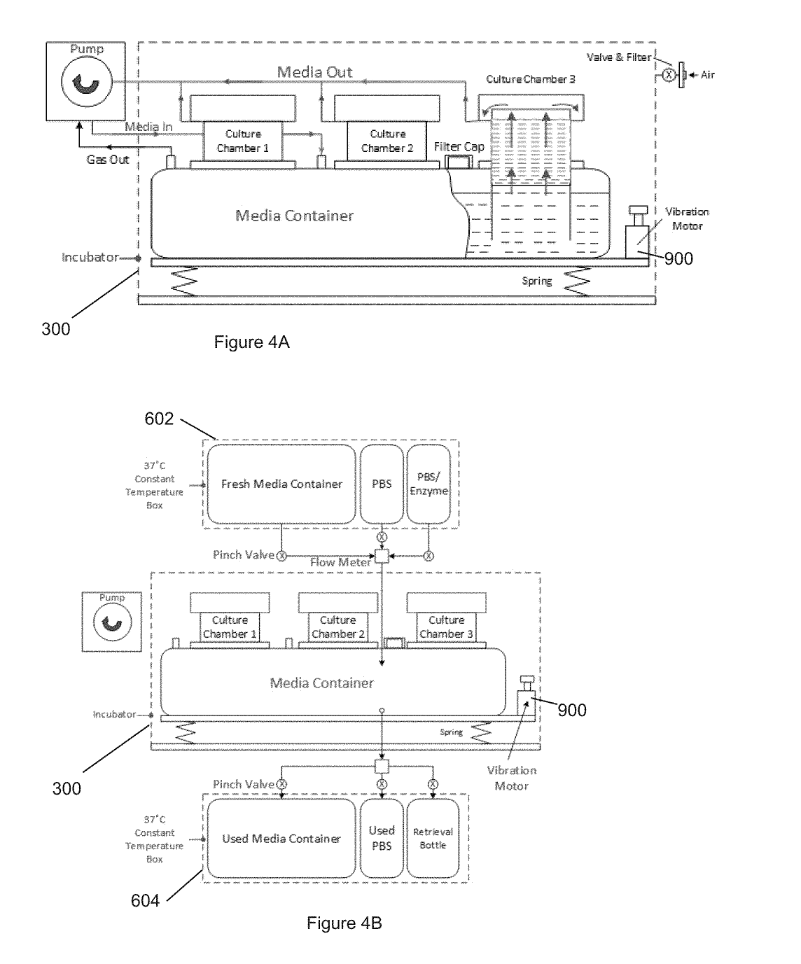

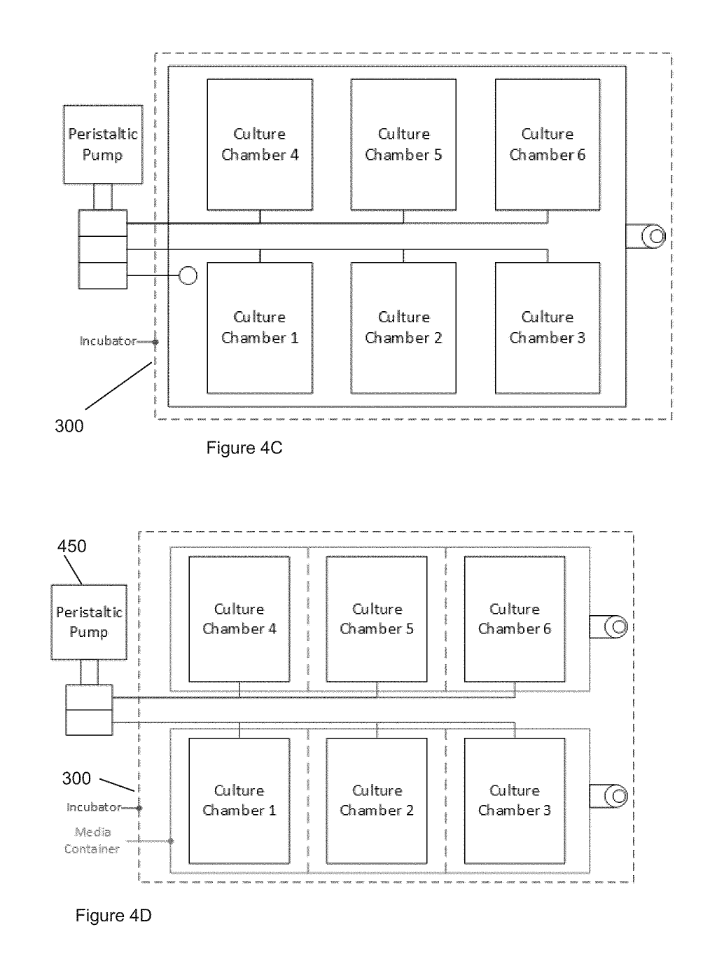

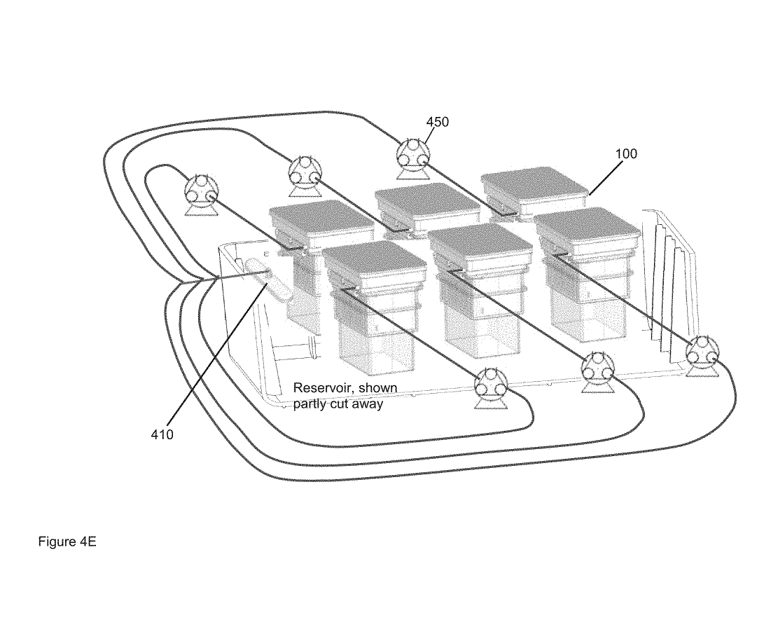

FIG. 4A is a side view, schematically, of a system showing three culture chambers (visible) sharing a common liquid pumping system.

FIG. 4B is another side view, schematically, of the system similar to FIG. 4A and additionally showing liquid storage containers above and below the central portions of the bioreactor system.

FIG. 4C is a top view, schematically, of the system having six culture chambers, with three of the culture chambers sharing a common liquid system and another three of the culture chambers sharing another common liquid system, and all of the culture chambers sharing a common reservoir.

FIG. 4D is a top view, schematically, of the system having six culture chambers, each in its own sub-reservoir, with three of the culture chambers sharing a common liquid system and another three of the culture chambers sharing another common liquid system and all of them sharing a common reservoir.

FIG. 4E is a three-dimensional view of a system having six separate liquid pumps but sharing a common reservoir.

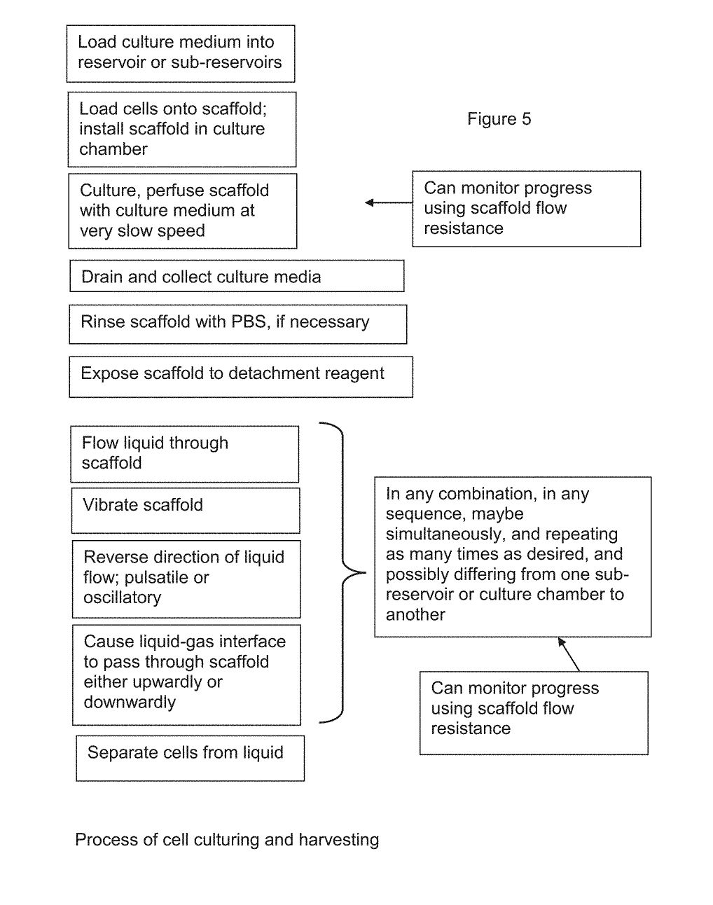

FIG. 5 shows a flowchart of a possible sequence of steps for culturing and harvesting of cells.

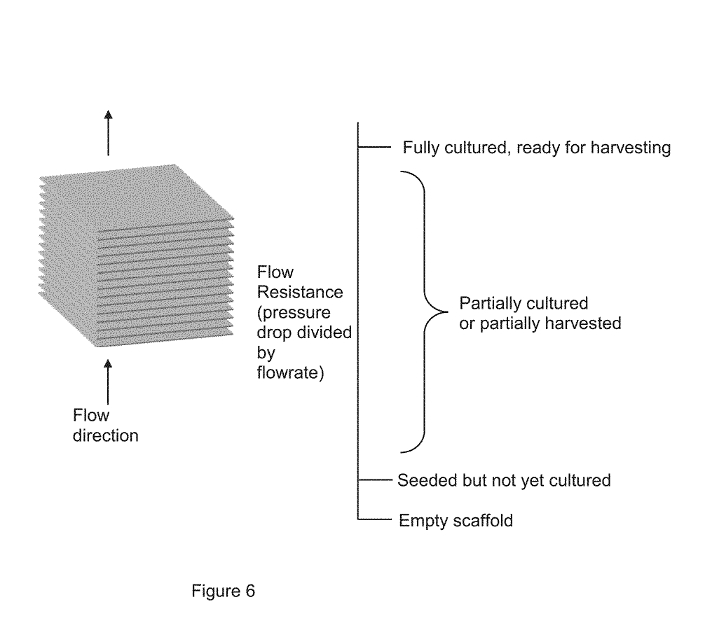

FIG. 6 shows a scale of flow resistance as might be encountered in using flow resistance to indicate number of cells present in a scaffold.

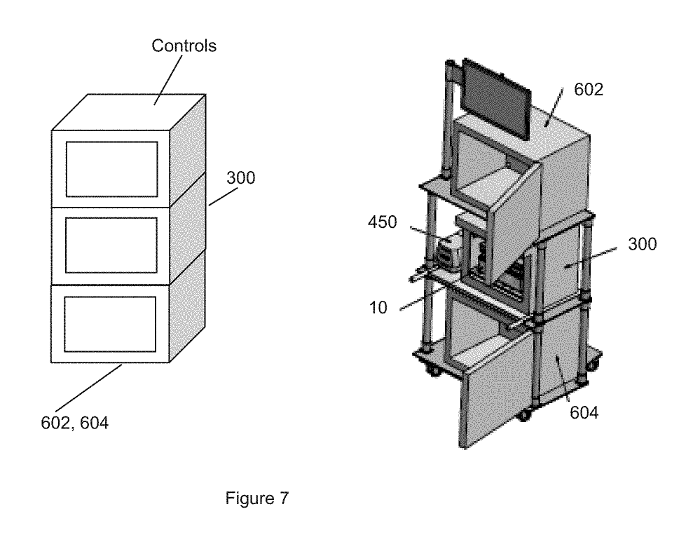

FIG. 7 shows possible physical arrangements of various components of the system.

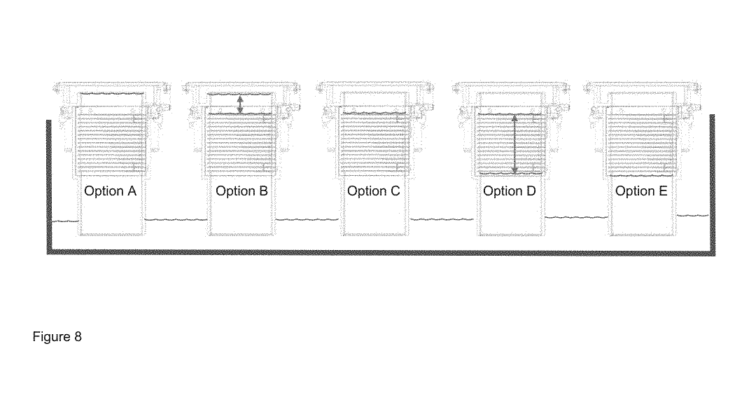

FIG. 8 shows positions and variations of a gas-liquid interface for various possible operations.

### DETAILED DESCRIPTION OF THE INVENTION

Referring now to FIGS. 1A and 1B, there may be provide a bioreactor 10 that contains an assembly of a cell culture chamber 100 and scaffold 110. Such an assembly is described in parent U.S. nonprovisional patent application Ser. No. 15/686,211, filed Aug. 25, 2017 and published as US20180057784. In such a bioreactor, cells may be cultured on a scaffold that is a crossed matrix of polymer filaments forming individual porous screens. The screens may be supported in a holder 120 that can hold a plurality (such as 12 to 15) of such screens in horizontal orientation, with the screens stacked vertically one above another. The holder 120 may be contained within a culture chamber 100. The culture chamber may be in communication with a reservoir 190. Above the stack of screens, the culture chamber 100 may include an open region surrounded by a weir wall 140 that has some open space above it. Outside the wall there may be a depressed region surrounding the wall, with the depressed region being referred to as a moat 160. The moat 160 may have a sump that is disposed at a vertically lower elevation than the moat 160 itself, and may have an exit connection 170 exiting from the sump. During culture, liquid medium may be perfused through the stack of screens, such as flowing in a vertically upward direction. During culture, when the liquid culture medium is flowing, there may be a trapped gas pocket that is located generally between the top of the weir wall 140 and the uppermost cover of the culture chamber, and which may also include some space within the moat 160. Depending on detailed dimensions and other design and operating parameters, a culture chamber as described therein, having typical practical dimensions, can culture approximately 250 million cells, if the screen contains four layers of filaments, and correspondingly more cells for larger numbers of layers of filaments.

Referring now to FIGS. 2A through 8, embodiments of the invention include a large-scale bioreactor system suitable for growing larger quantities of anchorage-dependent cells than are possible using only one of the just-described culture chambers. Such a bioreactor system can include a plurality of the just-described culture chambers 100. In an embodiment of the invention, as illustrated in FIGS. 2A-2B, the bioreactor system may comprise a reservoir assembly having six sub-reservoirs 200, with each sub-reservoir 200 having one culture chamber 100 in fluid communication with it. Other number of sub-reservoirs 200 and culture chambers 100 would also be possible. Sub-reservoirs 200 may collectively form a reservoir assembly. Such sub-reservoirs 200 and culture chambers 100 may be provided within a common incubator 300. Also, in such a system, the various culture chambers 100 and sub-reservoirs 200 may share use of certain common facilities such as controls and physical structure.

Having more than one sub-reservoir 200 within an incubator 300 provides that for certain parts of the overall system, such as the physical structure and the control apparatus and computer that controls various actions, it is only necessary to provide one of such component in the system, which has benefits in regard to economics and simplicity. At the same time, such an arrangement provides that within such a system there can be a plurality of liquid environments that are at least somewhat isolated from each other. With such an arrangement, if contamination accidentally occurs in one of the reservoirs or sub-reservoirs and its associated components in fluid communication with that reservoir, it is still possible that other reservoirs or sub-reservoirs and the associated components in fluid communication with those reservoirs could remain uncontaminated. Thus, it is possible that a single incidence of contamination might not render the entire contents of the overall system unusable.

With continued reference to FIGS. 1A-2B, there is shown a reservoir assembly that is an array of six sub-reservoirs 200 each having therein a culture chamber 100. The number six sub-reservoirs 200 is used for ease of illustration, and of course other numbers of sub-reservoirs 200 are possible. The sub-reservoirs 200 may be physically connected to each other and may share common walls separating adjacent sub-reservoirs 200. The assembly of sub-reservoirs may be topped by a cover 360. The cover 360 may contain openings therethrough so that a culture chamber 100 may be put in place, with the lower end of the culture chamber 100 extending down into a respective sub-reservoir 200.

The sub-reservoirs 200 may be in relation to each other such that at a lower elevation, each sub-reservoir 200 may be physically and fluid mechanically isolated from all other sub-reservoirs 200, but at an upper elevation the various sub-reservoirs 200 may be in fluid communication with some other sub-reservoirs 200. In embodiments of the invention, it may be described that during use, when liquid is present in the sub-reservoirs 200 up to a certain level, the liquid regions are isolated from each other, but the gas regions or headspace within a sub-reservoir 200 which are above the liquid regions, may be in fluid communication with the headspace of some other sub-reservoir(s) 200.

The assembly of sub-reservoirs 200 may be contained inside an incubator 300. Incubator 300 may be suitable to maintain a controlled temperature therewithin and also to maintain a desired composition of the gas contained therewithin. Similar to FIG. 2A, FIG. 2B shows an array of sub-reservoirs 200 inside an incubator 300. In FIG. 2B the culture chambers are omitted for clarity of illustration. Additionally shown in FIG. 2B are side-flow filters 380 that may be mounted in walls 384 that separate adjacent sub-reservoirs 200. The walls 384 that separate adjacent sub-reservoirs 200 from other sub-reservoirs 200 may have therein a side-flow filter 380 that allows gas to pass from one sub-reservoir headspace to an adjacent sub-reservoir headspace. The side-flow filter 380 may have sufficiently small pore size, such as 0.2 micron, so that it can prevent the passage therethrough of microorganisms.

It is possible that side-flow filters 380 are provided in some walls 384 but not in every possible wall. For example, as illustrated, side-flow filters 380 are provided in walls 384 between sub-reservoirs 200 that are in line with each other in one direction (the direction in which there are three sub-reservoirs in a row) but not in another different direction (the direction in which there are two sub-reservoirs in a row).

Referring now to FIGS. 3A-3E, at least some of the sub-reservoirs 200 may have a gas intake filter 390. As illustrated, all reservoirs have a gas intake filter 390. Through this gas intake filter 390, gas from the interior of the incubator 300 can pass to enter the headspace of the sub-reservoir 200. The gas intake filter 390 may have sufficiently small pore size, such as 0.2 micron, so that it can prevent the passage therethrough of microorganisms. For example, gas passing through the gas intake filter 390 can replace gas inside the headspace of the sub-reservoir 200 that may have become dissolved in the liquid as a result of liquid passing through the showerhead 410 and dripping downward back into the liquid region of the sub-reservoir 200.

FIGS. 3A-3E illustrate in more detail possible flowpaths of liquid and gas involving three sub-reservoirs inside an incubator 300. In FIGS. 3A-3D, the number of sub-reservoirs 200 is illustrated as three sub-reservoirs 200 simply for ease of illustration, and it can be understood that other numbers of sub-reservoirs 200 could be used similarly. All of the sub-reservoirs 200 are covered by a cover 360, which may be generally flat and horizontal in the illustrated orientation. Through the cover 360 a culture chamber 100 passes into each sub-reservoir 200, such that the lower end of the culture chamber 100 extends down to near the internal bottom of the sub-reservoir 200. An edge of the culture chamber 100 may rest upon the cover 360 and may form a seal with respect to the cover 360. The upper end of the culture chamber 100 extends above the cover 360. During operation, the lower end of the culture chamber 100 may be submerged in the liquid contained in the sub-reservoir 200. Although not illustrated, it is possible that a valve or filling/draining system may be provided to the sub-reservoir 200, suitable to allow the sub-reservoir 200 to be drained of or filled with appropriate liquid to a desired level within the sub-reservoir 200 and to allow such liquid to be replaced with a different liquid if desired.

In regard to the flow pattern of liquid during operation, as already illustrated, during cell culture in a culture chamber 100, the liquid culture medium may flow upward through the scaffold region and overflow the weir wall 140 into the moat 160. The moat 160 may have a sump into which the liquid from the moat 160 may further flow, and from the sump of each culture chamber 100, there may be tubing and a fluid flow path leading to a liquid pump 450. The liquid pump 450 may be a peristaltic pump or other type as appropriate. The outflow of the liquid pump 450 may return to the reservoir or sub-reservoir 200 that is in fluid communication with the same culture chamber 100. The return flow from the liquid pump 450 may re-enter the reservoir or sub-reservoir 200 through a showerhead 410 in the cover 360.

There is shown a gas exit from one of the sub-reservoirs 200, proceeding to a gas pump 480. The gas pump 480 may be a peristaltic pump. Peristaltic pumps are well suited to pump either liquid or gas. As a result of the side-flow filters 380, it is possible to remove gas from only one of the sub-reservoir headspaces, or to remove gas from less than all of the sub-reservoir headspaces, knowing that it is possible to have gas flow among sub-reservoir headspaces through the side-flow filters 380.

Among various culture chambers and sub-reservoirs, the liquid level in various sub-reservoirs can be chosen independently and can differ. The liquid such as liquid culture medium can be filled either manually or by a filling/draining pump which may be controlled by an automated system. The liquid or its composition can vary among various sub-reservoirs 200, if desired. The timing of operations such as filling and draining can differ from one sub-reservoir 200 or culture chamber 100 to another sub-reservoir 200 or culture chamber 100.

Referring now to FIGS. 4A-4E, in an embodiment of the invention, the culture chambers 100 may be either in fluid communication with a common liquid reservoir or in fluid communication with a sub-reservoir 200. The system may include any desired number of liquid circulation pumps 450. There may be a liquid circulation pump 450 dedicated specifically for each culture chamber, so that the number of liquid pumps 450 equals the number of culture chambers 100, or the system may include a liquid pump 450 dedicated to a subset of the plurality of culture chambers. The liquid pumps 450 may be capable of bidirectional operation and may be controlled by an automated control system. If there is more than one culture chamber 100 associated with a particular reservoir or sub-reservoir 200, for returning liquid to the reservoir or sub-reservoirs 200, there may be a common showerhead 410 by which flowpaths for all of the culture chambers, or for a subset of the plurality of culture chambers, come together and re-enter the reservoir by being dispersed as droplets above the liquid region of the reservoir. Such droplets, as they fall from the showerhead 410 to the liquid region of the reservoir, can exchange oxygen and/or carbon dioxide with the gas in the upper space (headspace) of the reservoir region. Alternatively, individual flowpaths and showerheads 410 could be provided.

FIG. 4A shows a system showing three culture chambers (visible) sharing a common liquid pumping system. The gas pumping system is shown as being driven from the same motor shaft as the liquid pumping system. FIG. 4B is another side view, schematically, of the system similar to FIG. 4A and additionally showing liquid storage containers for fresh liquids and used liquids above and below the central portions of the bioreactor system. FIG. 4C is a top view, schematically, of the system having six culture chambers, with three of the culture chambers sharing a common liquid pumping system and another three of the culture chambers sharing another common liquid pumping system, and all of the culture chambers sharing a common reservoir. The two liquid pumping systems and the gas pumping system are shown as all being driven from a single motor shaft, although of course it would also be possible to provide individual motors. FIG. 4D is a top view, schematically, of a system having six culture chambers, each in its own sub-reservoir, with three of the culture chambers sharing a common liquid system and another three of the culture chambers sharing another common liquid system and all of them sharing a common reservoir. FIG. 4E is a three-dimensional view of a system having six separate liquid pumps but sharing a common reservoir. Still further variations and combinations are possible in terms of the numbers of reservoirs, sub-reservoirs, liquid pumping circuits, and liquid pumps.

In some part of the system, an incubator 300 may provide a region that has a controlled temperature and also has an atmosphere that is controlled with respect to certain compositional variables, such as humidity and CO2 concentration. The interior of the incubator 300 may be clean or sterile. Inside the incubator 300 may be one or more reservoirs holding liquid, or one or more assemblies of sub-reservoirs 200. There may furthermore be one or more culture chambers that are in fluid communication with a particular reservoir or sub-reservoir. Each reservoir or sub-reservoir may be in fluid communication, as desired, with one culture chamber or with more than one culture chamber. The atmosphere inside the incubator 300 can be in fluid communication with the atmosphere inside a reservoir or sub-reservoir, as discussed elsewhere herein. There may be provided a gas intake filter 390 such that gas inside the incubator may pass through gas intake filter 390 to enter the headspace of a reservoir 190 or sub-reservoir 200.

In some part of the system (shown in FIG. 4B), there may be provided a region that is temperature-controlled but whose atmosphere is not controlled for any compositional variables. For example, a temperature-controlled region 602, 604 may be used to store liquid-containing containers or bags for which a certain temperature is desired.

Sensors, Controls and Software

In order to monitor relevant process parameters, the bioreactor system may comprise sensors for relevant parameters. Such parameters can be for pH, for Dissolved Oxygen and for other parameters of the culture liquid as may be desired. Another type of sensor that could be used is a sensor to measure glucose concentration or lactate concentration in the liquid. Concentration of carbon dioxide in gas in the incubator 300 or in the headspace of the reservoir or sub-reservoirs or the headspace of a culture chamber can also be measured. Such sensors may provide real-time data during the process, and can be used to adjust process variables such as composition, pumping speed of either liquid or gas, etc. The concentration of dissolved oxygen in the liquid culture medium could be used as an input to a control system so as to maintain the desired concentration by changing the concentration of the gas inside the incubator 300, such as by raising or lowering the concentration of oxygen or of nitrogen in that gas, in response to the measurement. Similarly, other measured parameters could be used to control process variables.

Additionally, in order to provide real-time visualization of cell growth, it is possible to install a miniature camera/video device on the top of the culture chamber in order to capture a snapshot of the cells on the scaffold at appropriate times. Another type of sensor that could also be used is a capacitive sensor that can measure or estimate the cell number. Any such sensors may be provided on any number of the culture chambers, ranging from one culture chamber to all of the culture chambers. Any such sensors can be used to control time duration of process steps.

Referring to FIG. 3A, there is illustrated a basic system having several culture chambers, sub-reservoirs and liquid pumps. As illustrated in FIG. 3B, it is possible that the sensor 700 can be directly in contact with liquid in the sub-reservoir. Alternatively, as illustrated in FIG. 3C, the sensor 700 can be connected to somewhere in the fluid flow circuit external to the sub-reservoir and can perform its sensing function somewhere external to the sub-reservoir.

It is possible that a sensor 700 may measure both dissolved oxygen and pH. Such sensor may penetrate through the top of the culture chamber into a particular sub-reservoir. Such a sensor may include a non-sterile multiple-use portion and a sterile one-time-use portion. The sterile one-time-use portion may essentially cover the non-sterile portion, and may prevent liquid in the sub-reservoir 200 from contacting the non-sterile portion. It is possible that a sensor based on measuring the electrical capacitance of the liquid in the sub-reservoir may be used to characterize the cells content of the sub-reservoir 200, which may in turn be used to estimate the degree of confluence of the culture that is in progress.

Another technique for estimating the number of cells within the scaffold, based on the scaffold's flow resistance, is described elsewhere herein.

In an embodiment of the invention, an imaging system may be installed on top of one or more culture chambers for real-time visualization of cell growth during the expansion process. The camera/video device, in particular, may help to determine the duration of the expansion process either in general for all of the culture chambers or specifically for one particular culture chamber, because populations of stem cells from different patients may grow at different rates and cells in different culture chambers could grow at different rates. It may be desirable for the expansion process to stop before the cells in the scaffold reach the state of confluency. The sensor device can communicate with the control software wirelessly through any of various communication protocols, including Bluetooth. Real-time images or video can be displayed on a computer screen.

These various sensors may be connected to a control system, which in turn, may connect and communicate with the software installed in the computer. Information from those sources may be used to control or adjust the culture conditions of the entire array of culture chambers. In another way of operating, information from those sources may be used to control or adjust the culture conditions of an individual culture chamber independently of what is done with other culture chambers in the system. Control or adjustment of the culture conditions may include any of: adjusting the flowrate of liquid medium through the scaffolds; adjusting the composition of the liquid medium; and choosing a time to end cell culture and begin harvesting. Depending on the number of liquid pumps 450 and the configuration of the tubing, adjustment responsive to the sensed information may be made for an individual culture chamber or a subset of the entire group of culture chambers or for all of the culture chambers 100. As a result, embodiments of the invention may have advantages over bioreactors currently available for the expansion of cellular products in regenerative medicine.

In regard to fluid flow arrangements, as discussed, the system may include a liquid circulation pump 450 dedicated specifically for each culture chamber, so that the number of liquid pumps 450 equals the number of culture chambers 100, or the system may include a liquid pump 450 that is dedicated to a subset of the plurality of culture chambers 100. Another possibility is that there could be liquid pumps 450 such as peristaltic pumps that contain a single motor but pump more than one channel of fluid. Alternatively, it is possible that instead of having an individual liquid pump 450 dedicated to an individual culture chamber, there could be adjustment of the proportioning of flow among culture chambers 100 achieved through valves such as proportional valves. Such valves could, if desired, divert flow of liquid medium to or away from particular culture chambers. Such adjustment could be done in response to conditions as measured by any of the sensors described herein. Any combination of such apparatus or techniques could be used.

In order to provide for data tracking and acquisition, a software program may be used for process control and data acquisition. All process parameters can be acquired at regular intervals and stored in a database for future reference and analysis. In addition, the software may also control the imaging functions and the automation steps for harvesting of cells.

In order to provide for an alert mechanism, the software can be programmed in such a way that an alert message may be sent appropriately when a certain critical parameter is out of range. For example, such an alert may be sent to the operator's cellular phone such as by using on-site Wi-Fi. This may enable timely corrective action to be carried out for abnormal operating conditions.

In order to provide for automated cell harvesting, a mechanism can be provided that provides for gently washing with saline such as Phosphate Buffered Saline (PBS) followed by washing with a harvesting reagent and optionally simultaneously applying a shaking motion. In such an automated mechanism, the flow of saline or harvesting reagent for rinsing the scaffolds may be controlled by the liquid pump 450 via the control software. A vibration mechanism such as a motor may be installed in mechanical contact with some part of the bioreactor system to aid in detaching the cells from the scaffold. The complete washing, detaching, vibrating and collecting cycles may be controlled by the control software.

### Determination of Extent of Cell Occupation by Pumping Characteristics

It is possible that the overall flow characteristics of the liquid flow circuit may be used to determine information related to the extent of presence of cells in the scaffold. As cell growth progresses, the number of cells in or on the scaffold increases, and also there can be an increase in the amount of extracellular matrix (ECM), which is material that is secreted by cells and exists in between cells. Both the cells and the ECM take up space within the scaffold. This reduces the space available for flow and increases the flow resistance of the scaffold. Flow resistance describes how much pressure drop is needed to achieve a given amount of fluid flowrate through the scaffold. Thus, the flow resistance can indicate how extensively the culture process has progressed and how close the culture is to confluence. Fluid resistance can be characterized from knowledge of pressure or pressure drop associated with the flow, together with a knowledge of fluid flowrate.

Generally, for such a characterization, it is helpful if the flow circuit contains a device to measure the pressure drop for flow of liquid through the stack of screens upon which cells are being cultured, or more generally to measure the pressure somewhere in the flow circuit. Such a pressure measuring device can be a pressure transducer 800. FIG. 3D illustrates a pressure transducer 800 connected to the fluid flowpath leading from the culture chamber to the liquid pump 450, in which case the pressure transducer 800 would be in communication with the liquid being pumped. Such pressure transducer 800 may measure the pressure at the point where it is connected to the liquid flowpath, which, when compared to ambient pressure, may provide a suitable pressure measurement. It is also possible (only illustrated in one place in FIG. 3D) that a pressure transducer 800 could be installed in the cover 520 at the top of the culture chamber 100, in which case the pressure transducer 800 would be in communication with the headspace (gas pocket) above the top edge of the weir wall 140. It would be possible to use a differential pressure transducer if the second side of the pressure transducer was connected to an appropriate place in the flowpath. It also is possible to use pressure measuring devices other than pressure transducers (such as pressure transmitters or other devices).

As discussed herein, the flowpath for liquid to perfuse through the scaffold may be driven by a liquid pump 450, which may be a peristaltic pump. Peristaltic pumps are suitable for both pumping the fluid and providing an indication of the volumetric flowrate of the fluid. Peristaltic pumps are substantially positive-displacement pumps, which means that the integrated flow is directly related to the integrated number of rotations of the pump motor, and the flowrate is directly related to the rotation rate of the pump motor. These flow parameters are also related to the dimensions of the pumptube of the peristaltic pump, which would be constant and known for any given apparatus. If the motor driving such a pump is a stepper motor, detailed information is readily available about the motor motion from the control system that operates the stepper motor. Yet another further possibility is that, even if no pressure measurement device is provided, the pressure can be inferred from the electrical power consumption of the motor. As the pressure drop across the flow circuit increases, the electrical power consumption of such a pump can be expected to increase similarly. It is thought a pressure transducer might provide a more accurate indication than would be provided by the electrical power consumption of the pump motor, but this would depend on individual circumstances.

The technique of using flow resistance to infer the degree of approach to confluence could be used to characterize the extent of cell growth during cell culture. It also could be used during the process of harvesting cells, in order to characterize how many cells have already been harvested and how many cells remain in the scaffold to be harvested. FIG. 6 conceptually illustrates the relationship of various measurements of flow resistance that may be taken during the processes described herein.

Similar to other feedback techniques described herein, the use of flow resistance as an indicator of extent of cells present in the scaffold could be used as a parameter to control or influence the process of either cell culture or cell harvesting. During cell culture, a measurement of flow resistance could be used to adjust parameters of the liquid culture medium, such as its chemistry or the duration of flow of the culture medium. During cell harvesting, a measurement of the flow resistance could be used to influence how long or how vigorously or with what combination of steps the harvesting process is performed. This can be advantageous in order to minimize the possible damage to cells resulting from various possible steps or aspects of the harvesting process. Such control could be performed individually for a particular sub-reservoir 200 or culture chamber 100, independently of what is done for other sub-reservoirs 200 or culture chambers 100. This enables the process parameters to be uniquely suited to a particular sub-reservoir 200 or culture chamber 100.

### Other Components and Physical Arrangement of Bioreactor System

### Auxiliary Tubing

There can be provided auxiliary tubing and pumps (not illustrated) to fill or drain liquid into or from individual sub-reservoirs 200. The liquid can be liquid culture medium, detachment reagents, or rinsing reagent such as Phosphate Buffered Saline. Such liquids can be handled in a way that prevents liquid from one sub-reservoir from ever coming into contact with liquid from another sub-reservoir 200 except possibly in a waste storage container. There can be independent pumps, or appropriate valving can be provided. Such practice can reduce the chance of possible contamination spreading from one sub-reservoir 200 to another sub-reservoir 200. Filling, draining and replacing of liquids from reservoir 190 or sub-reservoirs 200 can be performed under the control of the controller. The timing of such operations can vary from one sub-reservoir 200 to another sub-reservoir 200, as may be influenced by a sensor 700 as described elsewhere herein.

### Shaker

As is illustrated in FIGS. 4A-4B, there may be provided a shaker or vibration source for use in harvesting cells after expansion. The shaker or vibration source 900 may be in mechanical contact with the reservoir(s) or assembly of the sub-reservoirs 200, and may transmit vibration to the reservoir(s) or the assembly of sub-reservoirs 200. Parts of the apparatus may be mounted on springs or a cushion to assist in the management of vibration. The direction of vibration may be horizontal, or vertical, or other direction or combination of directions as desired. Operation of the shaker or vibration source may be controlled by the same controller or software that controls other functions of the system. Shaking or vibration may be performed during or shortly before certain steps of the harvesting operation.

### Physical Arrangement of System

Referring now to FIG. 7, in an embodiment of the invention, various components of the system can be assembled as illustrated.

The system can include an incubator 300 as already described, which may control the temperature of the culture chambers 100 and the reservoirs or assembly of sub-reservoirs 200 and may also control the composition of the atmosphere therein. The incubator 300 that surrounds the culture chambers 100 and reservoir or sub-reservoirs 200 may have an atmosphere therein, which may be controlled for any one or more of: concentration of oxygen, concentration of carbon dioxide, and humidity.

The system can also include a first temperature-controlled region 602 to control the temperature of fresh liquids waiting to be used. The system can also include a second temperature-controlled region 604 to control the temperature of containers that may contain substances such as used media, used saline solution, and a container that holds recovered cells.

As illustrated, the first temperature-controlled region 602 can be located at an elevation above the elevation of the incubator 300 and culture chambers 100 and reservoir and assembly of sub-reservoirs, so that gravity can drive the flow of liquids from the storage vessels into the reservoir 190 or sub-reservoirs 200. The second temperature-controlled region 604 can be located at an elevation below the elevation of the incubator 300 and culture chambers and reservoir, so that gravity can drive the flow of liquids from the reservoir to the containers that hold used liquids in the second temperature control region. Alternatively, for example for reasons of weight distribution and stability in the overall apparatus, as is also illustrated, it is possible to locate all of the storage containers (both fresh and used) at a relatively low elevation.

In the illustrated apparatus, fluids could be stored either in rigid containers or in bags. The use of flexible bags could more efficiently use the space inside temperature-controlled regions 602, 604, and flexible bags are widely used in medical applications and are inexpensive.

The system could also contain a computer or other control system, and pumps as required. The system could be assembled in a unitary cabinet and could be mounted on wheels. The motor of peristaltic pumps such as liquid pumps 450 or gas pump 480 could be mounted within the thickness of the wall of the incubator 300. The pump head itself could extend inside the incubator 300. Such an arrangement could reduce the length or complexity of tubing.

The system can also contain a separator apparatus that separates cultured cells from liquid. Such separator may be centrifugal, or may be a filter, or may be of other kind. Such separator could be mounted within the same apparatus as other components described herein, or could be a separate apparatus.

### Flow-Related Techniques Related to Cell Detachment and Harvesting

Embodiments of the invention include apparatus and techniques for harvesting cells from the bioreactor. Harvesting can involve a combination of any of various techniques including:
exposure to a detachment reagent;
rinsing out of the culture medium or the detachment reagent;
vibration or shaking in any desired direction;
flow of liquid through the scaffold, in a manner that may be either steady or intermittent or pulsatile or reversing direction of flow of liquid or oscillating;
passage of a liquid-gas interface past or through the scaffold.

It is believed that passage of a liquid-gas interface through the scaffold may serve to dislodge or detach cells from the scaffold. In embodiments of the invention, the culture chamber includes a headspace that typically during operation is a pocket of gas. FIG. 8 illustrates details of various possibilities for fluid motion and position of the gas-liquid interface.

In the fluid flow arrangements for flow of liquid as illustrated in FIGS. 3A-4E, the liquid may be culture medium, or detachment reagent, or phosphate buffered saline, or any other liquid as may be desired. These flow diagrams show that it is possible to operate a number of culture chambers independently of each other with various combinations of reservoirs sub-reservoirs and numbers of liquid pumps. In some of the illustrations, a plurality of culture chambers (three of them as illustrated) share a common reservoir. A showerhead 410 may be used with circulating culture medium, for the purpose of exposing the liquid culture medium to the CO2-rich gas that is inside the gas region (headspace) of the reservoir or sub-reservoir, so that the drops of the culture medium can absorb CO2 from that gas. If the culture apparatus is located inside an incubator 300, the interior of the incubator 300 may also be provided with that same CO2-rich atmosphere.

In embodiments of the invention the liquid pump 450 for pumping liquid through the liquid flowpath for various purposes. A liquid pump 450 for such purpose may be a peristaltic pump. For applications such as the present application, peristaltic pumps are positive displacement, are able to pump either liquid or gas, provide complete isolation of the fluid being pumped, and have a large base of experience. They also are able to move either the fluid in either direction depending on the direction of rotation of the pump rotor. If the liquid pump 450 is operated in the normal direction, liquid is withdrawn from the moat 160 and sent to the showerhead 410. If a peristaltic pump is operated in the reverse direction, gas can be taken in through the showerhead 410 and can be pumped into the moat 160. Specifically, the gas can flow into the sump in the moat 160, and then continue into the moat 160. If there is any liquid present in the sump or the moat 160, the gas can bubble up through whatever liquid may be present. Then the gas can pass into the upper region (headspace) of the culture chamber 100, which may be a gas pocket, and this may allow the liquid level in the culture chamber 100 to drop. As an alternative, the same effect could be achieved by opening an appropriate valve (not shown) in a branch of a Tee in the tubing that connects to the moat 160, and allowing the liquid level in the culture chamber 100 to drop as gas is introduced into the tubing. It is possible for the process to be repeated and alternated so that the liquid-gas interface passes through the culture chamber 100 and scaffolds 110 up and down repeatedly at a desired velocity.

It is possible that, while the culture chamber 100 contains liquid and the scaffolds 110 are and remain immersed in liquid, the direction of liquid flow through the scaffolds and the culture chamber can be reversed and alternated. This would produce a liquid velocity flowing past the scaffolds 110, in the vertical direction, that alternates its direction. If it is desired that such flow reversal takes place while all the scaffolds remain submerged, there may be provided, within the culture chamber, a sufficient space that is located, in a vertical sense, between the uppermost surface of the uppermost scaffold 110 and the top of the weir wall 140. Within such space, the liquid level can rise and fall as desired in order to accomplish the two opposite flow directions for liquid flow in the vertical direction through the scaffolds 110.

If there are a plurality of culture chambers 100, it is possible that during any period of time, there may be flow of appropriate liquid (culture medium, harvesting reagent, rinse) vertically upward simultaneously through all of the culture chambers. This will involve the liquid occupying a level up to the top of the weir wall 140 (overflow wall) in each of the culture chambers 100. In such a situation, the amount of liquid required will be at least enough to fill the interior of each culture chamber from its bottom edge to the top of the weir, plus a volume to keep the reservoir level at least up to the bottom edge of each culture chamber 100.

In embodiments of the invention, it is possible for there to be any of various different liquid levels in particular culture chambers 100 as desired. Furthermore, it is possible that in a system of an embodiment of the invention, containing a plurality of culture chambers 100, at any given time, different culture chambers 100 might be operated in different ways among the options described herein. Any such operations could be performed at different times in different culture chambers 100. In whichever of the culture chambers 100 this may be desired, the liquid level can be time-varying. Various options are illustrated in FIG. 8. In FIG. 8, a wavy line indicates an interface between liquid and gas. In options where two such interfaces are shown with a double-ended arrow between them, the illustration illustrates that the liquid-gas interface can move back and forth between the two illustrated locations of the interface. Several such options are shown. Outside the culture chambers 100, a generic liquid level is shown for a common reservoir, but it can be understood that the culture chambers could be associated with individual reservoirs or sub-reservoirs.

Referring now to FIG. 8 Option A, it is possible that, for a particular culture chamber, with all of the scaffolds 110 being submerged in liquid, the liquid could be to the top of the weir wall 140 and could remain that way for an extended period of time. There could be continuous flow of liquid in an upward direction, such that all of the scaffolds 110 are submerged and there is continuous overflow of liquid over the weir wall 140. It is also possible for the liquid to be static with the gas-liquid interface being at the top of the weir wall 140. This can occur during cell culture, when the liquid is culture medium. It also could occur at certain stages of harvesting and recovery of cells, such as perhaps later stages of that process. In such a situation the liquid could be any of various liquids.

Referring now to FIG. 8 option B, it is possible that, with all of the scaffolds 110 being submerged, a culture chamber 100 could use oscillating or variable-velocity flow of liquid past or through the scaffolds. This could be done in order to help detach cells from the scaffold by the shear stress of the flowing liquid. It is possible that during such a procedure, the liquid level in the culture chamber can be somewhere between the top of weir wall 140 and the upper surface of the uppermost scaffold. That liquid level can vary as a function of time. If the liquid level in a particular culture chamber 100 varies in an oscillatory manner, that would be associated with alternating directions of flow of liquid through the scaffolds, and hence alternating direction of shear stress experienced by the cells. Pulsatile waveforms of flow could also be provided. That situation could also be useful for detaching cells.

Referring now to FIG. 8 Option C, another possibility is that that, with all of the scaffolds in a particular culture chamber being submerged in liquid, in that culture chamber the liquid level may be maintained above the level of the uppermost scaffold, and may be maintained in a static situation.

Referring now to FIG. 8 Option D, it is possible that the liquid-gas interface could be somewhere within the scaffold region or could pass through the scaffold region in a time-dependent manner. For a culture chamber in which motion of the liquid-gas interface is used to help detach cells from the scaffold, it is possible that the liquid level can be below the bottom of the lowest scaffold at certain times, and could be above the top of the uppermost scaffold at other times, or alternatively could be somewhere within the scaffold region. That liquid level can vary as a function of time. It is possible that the time-varying position of the liquid-gas interface could be helpful for detaching cells from the scaffold.

Referring now to FIG. 8 Option E, still further, it is possible that there may be a culture chamber in which all of the scaffolds may be exposed to gas for a defined period of time, i.e., the liquid level may be below the bottom of the lowest scaffold and may be stationary for a period of time. This situation may be used in order to reduce the overall amount of liquid that is needed, especially if the liquid is expensive.

Any of these options could be performed with any liquid of interest (such as culture medium, rinse, or harvesting reagent) in the culture chamber. The operation of the system according to Options A-E can be controlled by the operation of individual liquid pumps 450, including their pumping speed and direction of flow. Peristaltic pumps are one possible type of pump. As illustrated in FIG. 3A, each culture chamber 100 may be associated with a dedicated liquid pump 450 for pumping liquid in the liquid path of that particular culture chamber. Other arrangements (involving different numbers of liquid pumps 450 in relation to the number of culture chambers) are also possible.

During the harvesting process, the use of vibration applied by the shaker or vibration source 900 may be coordinated with particular features of the motion of liquid or the liquid-gas interface. For example, if the vibration is in a vertical direction, and if there is vertical velocity of liquid while the culture chamber is submerged in liquid, it is possible that motion of liquid in the vertical direction can superimpose on vertical motion due to vibration to increase forces acting to detach cells. Also, if the vibration is in a vertical direction, and if there is a liquid-gas interface that moves up or down past a screen, it is possible that motion of the liquid-gas interface in the vertical direction can superimpose on vertical motion due to vibration to increase forces acting to dislodge or detach cells. It is also possible that vibration could be in a horizontal direction or other direction.

If liquid flow is performed in an oscillatory manner, there is a frequency of flow oscillation, and if vibration is applied, there is a frequency of vibration. It is possible that the two frequencies could be different from each other, such as the applied vibration frequency being faster than the liquid oscillation frequency, with there not necessarily being any particular relationship between the frequencies. It is possible that the frequencies could be chosen such that one of the frequencies is an integer multiple (harmonic) of the other. It is possible that the frequencies could be chosen to be equal to each other. In that situation, or in a harmonic situation, there could be a relative phase relationship as desired between the fluid oscillation and the mechanical vibration. For example, the fluid flow oscillation and the applied mechanical vibration could be phased such that the maximum force on cells caused by the fluid flow oscillation and the maximum force on cells caused by the applied mechanical vibration could be simultaneous in time and in the same direction, so as to create a combined peak force acting to dislodge cells. This could be especially true if the direction of vibration is vertical similar to the direction of fluid motion. It also is possible that the vibration could be intermittent even if the fluid oscillation is continuous, or vice versa.

### Methods of Culturing and Harvesting

An embodiment of the invention can include a method of culturing and harvesting cells. Such method can include providing a bioreactor system as described herein, having a plurality of culture chambers and a plurality of sub-reservoirs and a plurality of circulating liquid pumps 450, and operating various culture chambers independently or differently from each other. Such method is illustrated in the flowchart of FIG. 5.

In the method, cells can be seeded onto scaffolds using an apparatus such as the apparatus described in one of the U.S. provisional patent applications that is incorporated by reference herein. Alternatively, it is possible to seed cells by hand using pipettes or similar apparatus. Cells could be seeded on an individual scaffold or screen in a uniform spatial distribution within the scaffold or screen. Alternatively, if desired, they could be seeded in a spatial distribution that is non-uniform within the scaffold or screen. If cells are seeded by an automated system, any distribution could be programmed by associating a particular amount of cell deposition with a particular location of the dispenser. The various scaffolds or screens (such as 12 to 15 of them within a culture chamber as described) could be seeded identically to each other. Alternatively, some of the scaffolds or screens could be seeded in patterns that are different from the patterns of other scaffolds or screens. The scaffolds and a scaffold holder, containing seeded cells, can then be loaded into the culture chambers, which can then be assembled together with the reservoir or sub-reservoir.

At the beginning of the culture process, fresh fluids can be loaded into the first temperature control region and can be brought to the desired temperature. Then, a desired quantity of liquid culture medium can be allowed to flow or can be pumped into the reservoir or sub-reservoir. When the liquid culture medium and the seeded scaffolds are present, flow in individual culture chambers can be initiated to perfuse through the scaffolds so as to provide nutrients during culturing. This perfusion can continue for a desired time, which may be approximately one week or more. As described herein, the bioreactor system may comprise at least one sensor 700 and possibly could even comprise sensors 700 for individual culture chambers, and may be able to sense any of several parameters that are relevant to the cell culturing process. The sensors 700 may interact with the control system to adjust or control the operating parameters of the system or of an individual reservoir or sub-reservoir or liquid flow circuit or gas composition. For example, whatever liquid pumps 450 are present, which could be as many liquid pumps 450 as there are culture chambers 100, could be operated independently of each other such as by being responsive to particular sensors.

If a particular liquid pump 450 is operated in a forward direction, it can circulate liquid culture medium through its flowpath, flowing upward through the scaffolds, as described elsewhere herein or in documents incorporated by reference. Forward flow of culture medium through a particular culture chamber can be performed for as long as desired for culture to occur. This time duration may be responsive to conditions measured by any one of the sensors or camera. Also, the flow velocity could be responsive to conditions measured by any one of the sensors or camera. Also, measurement of the flow resistance of the scaffold, as described herein, could provide an indication of the number of cells attached to the scaffold, and this could be used to determine operating parameters or the duration of culturing. These operating parameters could differ among the various culture chambers.

If a particular liquid pump 450 is stopped in the just-described condition, there can be static condition of liquid, such as culture medium, surrounding the scaffolds. Such static condition can be with the culture chamber filled with liquid up to the top of the weir wall 140.

If such liquid pump 450 is operated in a reverse direction, for a short while it can cause liquid culture medium to flow in a reverse direction, corresponding to downward flow of liquid culture medium through the scaffolds. It also is possible for the liquid pump 450 to be stopped either with the liquid level being above the uppermost scaffold or with the liquid level being within or below the region where the scaffolds are.

There is a certain volume of tubing that extends from the moat 160 through the liquid pump 450 (which may be a peristaltic pump) to the showerhead 410. There also is a certain volume of the moat 160 as defined by space from the bottom surface of the moat 160 to the top of the overflow weir wall 140 that defines the moat 160. If the liquid pump 450 has been operating in the forward direction for some time, it can be expected that the tubing is full of liquid. It also is typical that the liquid level in the moat 160 is fairly low, i.e., close to the bottom of the moat 160. It may be desirable that when the direction of flow in the tubing is reversed, the liquid pump 450 may operate so as to introduce gas entering the tubing from the showerhead 410. This gas may bubble up through the liquid in the moat 160 or the sump connected to the moat 160. The intent may be that the gas eventually reaches the headspace in the culture chamber 100. At the same time as gas is entering the tubing near the showerhead 410, liquid is displaced from the tubing near the moat 160 back into the moat 160. It may be desirable that when liquid is flowing back into the moat 160, the moat 160 should not overflow liquid back onto the scaffolds. This can be accomplished if the internal volume of the tubing between the showerhead 410 and the moat 160 is less than the volume of the moat 160. If a sufficient volume of pumping in the reverse direction is done, it can be expected that the liquid from the tubing will go back into the moat 160, and upon further pumping gas will be moved from the showerhead 410 into the upper space of the culture chamber 100, which will involve the gas bubbling up through the liquid in the moat 160 and the sump connected to the moat 160, and the pumped gas will displace liquid in the culture chamber allowing the liquid level in the culture chamber to drop.

Next, if the cell culture period is finished for a particular culture chamber or for all of the culture chambers 100, the liquid culture medium can be drained from the reservoir or sub-reservoirs. As desired, for all of the culture chambers, the fill pump or pumps (not illustrated) can be operated to empty the culture medium out of the various tubings. For the next operation, the reservoir or sub-reservoirs can then be filled with saline such as Phosphate Buffered Saline, or with an detachment reagent which may be in Phosphate Buffered Saline, or one of these liquids at one time and another at another time. If the liquid culture medium contains serum, it may be desirable to rinse the culture region with a rinse liquid such as saline, before introducing the detachment reagent. If no serum is present, it might not be necessary to perform a rinsing step. Then, the fill pump(s) can be operated to fill the culture chambers with that liquid as desired. It is possible that all of the culture chambers can be filled with the liquid simultaneously and flow of the liquid can occur through all of the culture chambers simultaneously. It is expected that harvesting only requires exposure of the scaffolds to detachment reagent for a short period of time such as 15 minutes. During this period, the flow of detachment reagent or other liquid can be steady or intermittent or oscillatory or pulsatile or can have reversals of direction of flow, as may be desired, as discussed elsewhere herein.

After the desired duration of exposure of the scaffolds to detachment reagent, the detachment reagent can be drained or pumped out from an individual culture chamber. After all of the culture chambers have been exposed to detachment reagent, the detachment reagent can be drained from the reservoir. If desired, the reservoir can again be filled with a reagent such as Phosphate Buffered Saline.

It is possible that during harvesting, the liquid pump 450 can be operated in a steady flow mode, similar to what was done during culturing. However, in this situation, the flowrate and liquid velocity through the scaffolds can be chosen to be appropriate for harvesting, which may be different from (larger than) what was used during culturing.

It also is possible that the liquid pump 450 can be operated in a pulsatile or time-varying mode, such that even if flow of liquid is in a single direction for extended periods of time, the flowrate or velocity can vary. Pulsatile flow could be understood as having a brief burst of velocity or flow in a particular direction, and also a period of lesser flow in the same direction, but with an overall waveform that is different from the typical sinusoidal waveform. It is possible that brief bursts of larger-than average velocity of liquid, even if followed by less-vigorous conditions, could dislodge or detach cells, and the lower-velocity or less-vigorous conditions could serve to transport detached cells out of the culture chamber.

It also is possible that liquid flow could be operated in an oscillating manner. In n oscillating flow mode, the liquid flow direction could change repeatedly, and the volume of liquid displaced during any one oscillation could be relatively small, as could the distance that a given segment of liquid moves through the scaffold during oscillation. Such a situation could be produced, using a peristaltic pump, if the rotor of the peristaltic pump rotates back and forth alternating its direction of rotation. Such oscillation could be sinusoidal but does not have to be.

It is possible that flow regimes could be performed in one culture chamber 100 and sub-reservoir 200 in a manner or sequence that is different from what is performed in another culture chamber 100 or sub-reservoir 200.

It is also possible that the liquid pump 450 can be operated in alternate directions for a small amount of volume displacement while the scaffold region is still fully submerged in liquid. This can cause alternating up and down flow of liquid past the scaffolds, which may be appropriate for dislodging cells from the scaffolds. It would also be possible to combine, in some sequence, the just-described alternating flow with the just-described pulsatile flow. For example, some reverse-direction flow of liquid could be followed by forward-direction flow of liquid in a relatively strong velocity or flowrate, which could be followed by a period of more gentle liquid flow. Any of this could be simultaneous with externally imposed vibration as may be desired. The frequency of the oscillation of the flow could be different, even significantly different, from the frequency of vibration; alternatively, if desired, the frequency of the oscillation of the flow could be the same as, or almost the same as, the frequency of vibration. In the latter situation, the vibration and the flow oscillation could be adjusted to be in-phase with each other, in a way such that accelerations experienced by the cells due to vibration could reinforce forces experienced by the cells due to liquid motion. However, this is not essential.

It is further possible that in this situation, the liquid pump 450 can be operated so as to cause a liquid-gas interface to pass through the scaffold region, perhaps repeatedly. The liquid pump 450 can be operated first in one direction and then in the opposite direction, displacing a volume of gas appropriate to change the liquid level in the culture chamber from one position to another so as to alternately expose and submerge the scaffolds in liquid.

Still further, it is possible that for a given culture chamber 100 and scaffold and sub-reservoir, a determination could be made as to the progress of harvesting, based on the flow resistance of the scaffold at any given time during the harvesting process. This could be done, for example, when the scaffold is submerged in liquid. The progress of the harvesting process can be estimated by observing the flow resistance (or the change in flow resistance) of the scaffold as a function of time during the harvesting process. The flow resistance of the scaffold can be characterized in generally the same way that has been described herein in connection with estimating the degree of cell growth (approach to confluency) during the culturing process, by using pumping-related information.

For example and for reference, as illustrated in FIG. 6, it would be possible to characterize the scaffold flow resistance for several relevant situations. One would be an empty scaffold, with no cells located on it. Another would be a scaffold at the very beginning of cell culturing, after cells have been seeded onto the scaffold but before cell growth has occurred. Another would be the scaffold at the time when it is decided that cell culture should end and harvesting should begin. There are also intermediate situations which could be characterized. When a scaffold is partially cultured, it would have a flow resistance somewhere between the value at the beginning of culture and the value at the end of culture. When a scaffold is partially harvested, it would have a flow resistance somewhere between the value at the beginning of harvesting and the value for a completely empty scaffold.

For example, if the scaffold is close to confluence at the beginning of harvesting, the scaffold would have a relatively large flow resistance, which would be reflected in the pressure drop. The flow resistance can be determined from a calculation using the liquid flowrate and the pressure drop. At a later stage during harvesting, when some of the cells would likely have been removed, the flow resistance of the scaffold would likely be smaller. This information could be used to determine how long the harvesting process should continue. There is potential for the harvesting process to damage cells, so it is advantageous that the harvesting process not continue longer than necessary. Similarly, this information could be used to adjust what technique is used at a given time during the harvesting process. It is possible that one technique such as steady flow might be more useful at a certain stage of the harvesting process, and another technique such as alternating or pulsatile flow or passage of a gas-liquid interface could be more useful at another stage of harvesting, and this measurement of flow resistance of the scaffold could be an indicator of what is the stage of harvesting and hence what is the most appropriate technique to use at that time. This indicator of harvesting can be done uniquely for each culture chamber 100, or for each culture chamber 100 that is pumped by a dedicated liquid pump 450 that has pressure measurement instrumentation somewhere in the flowpath or the culture chamber 100.

Such pressure transducer 800 may be located between the liquid pump 450 and the culture chamber 100. As illustrated, the pressure measured may be sub-atmospheric, but that can be taken into account by the pressure transducer and associated software.

In connection with such a situation, it may be desirable that where the individual tubings come into the showerhead 410, they not join with each other upstream of the showerhead 410, so as to avoid the possibility of one of the tubings sucking liquid from the other tubings if the liquid pump 450 for that particular tubing is being operated in reverse while other liquid pumps 450 are being operated in a forward mode. Also, it would be possible to have separate showerheads 410 for each sub-reservoir 200.

Given the existence of individual liquid pumps 450 for individual flow circuits, or the ability to operate individual flow circuits differently, it is possible that harvesting operations in various culture chambers can be carried out non-simultaneously. For example, if one culture chamber is ready for harvesting earlier than another culture chamber, harvesting operations can be performed on it at an appropriate time irrespective of what is taking place in another culture chamber. This can be a function of how close the cells in a particular culture chamber are to reaching confluence.

When cells are being harvested, it may be desirable for some of the flow to be vertically downward through the scaffold followed by an opportunity for cells to settle out of the liquid into or towards the bottom of the respective reservoir or sub-reservoir 200. It is expected that the harvested cells have a density greater than the density of the various liquids that may be caused to flow through the apparatus, and so the cells will tend to sink out of the liquid down to the bottom of the reservoir or sub-reservoir 200. During the harvesting process, appropriate pauses and duration of static conditions can be provided for this to occur. It is believed that this is preferable compared to causing harvested cells to flow through the peristaltic pump 450 and the showerhead 410.

After harvesting, the liquid contained in the reservoirs or sub-reservoirs 200 can be subjected to a procedure that separates the harvested cells from the liquid. This can be done by centrifugation, filtering, or other appropriate processes. It is further possible that the harvested cells can be rinsed, such as with saline (Phosphate Buffered Saline) in order to remove detachment reagent that might remain on the cells. It is also possible to perform tests to determine the effectiveness of rinsing and removing the detachment reagent from the cells.

In some cases, it may be desirable that the recovered cells be stored by being frozen. In such a situation, the recovered cells can be re-suspended in a solution adapted for freezing, and can then be subjected to appropriately low temperatures to freeze the cells. Cells can be stored, for example, in liquid nitrogen.

In still other applications, it may be that what is of value from the cell culturing process is proteins that are secreted by the cells during culturing. In such processes, the cultured cells themselves might not be of value. In such a case, there would be no need to apply detachment reagent or to perform any of the other steps associated with harvesting.

### Operating Different Culture Chambers Differently During Harvesting

Bioreactors can be monitored for any of various process parameters associated with their operation, including but not limited to: pH of the culture medium; temperature; concentration of glucose in the culture medium; concentration of lactate in the culture medium; concentration of dissolved oxygen in the culture medium; concentration of carbon dioxide in the atmosphere above the liquid; numbers or confluence of cells growing on substrates. It is also possible that any of these could be used as a parameter to control a feedback loop that would adjust a process parameter to achieve a desired result.

As described herein, there could be provided a plurality of sub-reservoirs each having a culture chamber associated therewith. It is possible that for each culture chamber there can be a dedicated fluid flow circuit that moves liquid culture medium past the scaffolds during culturing. Such circuit can have individual control of fluid flowrate, such as by an individually controlled liquid pump 450. In response to the conditions as indicated by a sensor, it is possible to adjust any one or more of the following during either cell culturing or cell harvesting: volumetric flowrate of liquid; duration of liquid flow; direction of liquid flow.

In particular, for the described culture chamber that comprises a weir above the scaffold region and during operation contains an air pocket, it is possible to cause a gas-liquid interface to move past the scaffold region in either the upward or downward direction as desired, at a desired velocity and a desired number of repetitions.

Any of these harvesting operations could be done differently for different culture chambers, and may be done responsive to sensed values of any of the described parameters. For example, harvesting operations do not have to be performed simultaneously for all of the culture chambers; rather, harvesting operations could be performed when a determination is made that for that particular culture chamber, an appropriate level of progress toward confluence has been reached. Also, the duration of harvesting operations does not have to be identical for all of the culture chambers 100.

With appropriate fluid connections, liquid culture medium can be removed and replaced with harvesting liquid.

A detachment reagent can contain reagents such as enzymes that loosen the attachment of cells to neighboring cells or to the substrate. An example of such a harvesting enzyme is trypsin. Another is collagenase. It is further possible that either in combination with any of these enzymes, or alone, the liquid flowed during cell detachment or harvesting could contain additives such as surfactants, or a triblock copolymer that helps reduce damage to cells by harvesting enzymes, or similar substances. An example of such a triblock copolymer is a triblock copolymer polyoxyethylene-polyoxypropylene-polyoxyethylene, commercially available as Pluronic®, available from BASF Corporation. More specifically, a suitable member of that family is Pluronic F-68. Pluronic F-68 has an average Molecular Weight of about 8400 Da, of which ethylene oxide makes up approximately 80%. Pluronic is believed to protect cells from externally applied shear stress, by reducing the effect of shear stress applied to the cells. It is also possible to include a surfactant either alone or in combination with other substances mentioned herein. The liquid flowed during cell detachment or harvesting can be aqueous having a surface tension of less than 50 dynes/cm, or less than 40 dynes/cm, or less than 30 dynes/cm.

A sensor 700 could be a probe that touches the liquid in the sub-reservoir, as illustrated in FIG. 3B, or it could be a probe somewhere else in the fluid flow circuit such as in the tubing that goes back and forth to the liquid pump 450, as illustrated in FIG. 3C.

Alternatively, it is possible that the culture chambers associated with a group of the sub-reservoirs (while still not being all of the culture chambers) could be controlled together.

In order to achieve detachment of cells, it is only necessary that the scaffold be exposed to the detachment reagent for a relatively short amount of time, such as approximately 15 minutes. That is not very long (compared to the typical culturing time of approximately one week). It is a matter of preference as to whether the exposure to the detachment reagent is simultaneous with the rocking or with vibrating of the scaffold or with certain flow regimes as described elsewhere herein. It would be possible to fill one culture chamber with detachment reagent, possibly including vibrating it for the appropriate period of time, while the other culture chambers do not contain detachment reagent.

It is further possible that there could be sub-reservoirs 200 or a reservoir that is subdivided into sub-regions that are plumbed and controlled separately. In such a situation, it is possible that while one culture chamber is exposed to detachment reagent, the other culture chambers could still contain culture medium as they do during the duration of the culture process. This could be determined by process parameters as measured for individual culture chambers 100. Many combinations of different conditions in different culture chambers 100 are possible, as described elsewhere herein.

It is also possible that there could be provided a plurality of sub-reservoirs as described and illustrated, and within a sub-reservoir there could be provided two or more culture chambers sharing the same sub-reservoir, rather than just one culture chamber per sub-reservoir as has been illustrated.

### Additional Comments

As described herein, within the culture chamber 100 there may be an overflow weir wall 140 defining a moat 160 with an exit at a lower elevation than the top of the overflow weir wall 140, such that when in operation, there is a trapped volume of gas above the liquid that is inside the culture chamber 100. However, the presence of a trapped volume of gas is not essential, and as an alternative it is also possible to operate a culture chamber 100 in a mode in which the interior of the culture chamber 100 is completely filled with liquid.

If the culture medium contains serum, it may be desirable to rinse the scaffold with a rinse such as saline phosphate buffered saline before use of harvesting reagent. If serum-free culture medium is used, it may be unnecessary to rinse the scaffold,

The described method of monitoring extent of cell growth and also extent of cell harvesting by characterizing the flow resistance of the scaffold may be advantageous for monitoring those parameters, especially because the method is relatively non-invasive. It does not require disassembling any portion of the system to obtain a measurement, and can be performed continuously, and if a sensor or monitor is connected to tubing, does not even require a sensor or monitor to penetrate the boundary of the culture chamber itself. This also can be done uniquely for a particular culture chamber.

On such scaffolds, the 3D printed surface provides a three-dimensional surface area for growth, thereby providing much more area available for cell expansion as compared to a similar flat culture plate. For a given volume, it is possible to pack more than 5-7 times more cells on such scaffolds than on a comparable flat plate. This number can be increased as needed, depending on cell type, by adjusting the spacing density of the fibers. The fact that rich ECM (Extra Cellular Matrix) can be developed across the pores or spaces between the fibers of the scaffold can provide additional area for cell growth.

The 3D printed nature of the scaffold on which the cells expand and grow can be digitally defined. It is possible to control the spacing, the pattern, and the fiber diameter to change various expansion parameters, such as the surface area available for cell attachment/growth, easier flow of culture media through the scaffolds, by either increasing or decreasing the pore sizes or spacing in the scaffold.

Because the surfaces are grid-like 3D printed surfaces, the process of removing cells at the end of the expansion process is significantly easier than in the case of cell culture technologies such as hollow-fiber bioreactors or bioreactors that use micro-particles or micro-carrier as culture surfaces. Because the pores of the described scaffolds are well defined, the flow parameters of systems of embodiments of the invention may be chosen to allow easy retrieval of cells. This differs from cell culture using micro-particles, in which extensive use of enzymes and time is needed to extract the cells, especially mesenchymal stem cells.

Because in embodiments of the invention the 3D printed scaffolds are stationary, the only shear stress experienced by the cells or scaffolds during cell culture is due to the flow of culture media past the scaffold surface. Therefore, it is easier to control and adjust the shear stress experienced by the cells. In contrast, in bioreactors that use micro-particles as scaffolds, the shear stresses experienced by cells are not easily modulated because the microspheres rotate within the vessel as they bounce around, which makes it almost impossible to model and control the shear stress levels. In embodiments of the present invention, the shear stress experienced by cells in the bioreactor is consistent across all surface areas that are available for cell growth.

The use of polystyrene as the material of 3D printed scaffolds makes use of existing experience, because polystyrene is a material used frequently in tissue culture plates for anchorage-dependent cells.

Because of the interconnectedness of the empty spaces in the scaffolds described herein or in documents that are incorporated by reference, it is expected to be possible to detach and collect over 90% of the cells after expansion.

Compared to other currently available technologies for cell culture, embodiments of the invention are a closed system, easier and less expensive to operate, requires less maintenance and is more automated than currently available system.

For some applications it is desired to harvest and make use of the cultured cells themselves. However, it is not always necessary to harvest cells from a bioreactor. There are some other applications in which the secretions of the cells are of interest, rather than the cells themselves.

What is referred to herein as saline solution could be Phosphate Buffered Saline.

Pumps (either liquid pumps or gas pumps or fill/drain pumps) can be peristaltic pumps or other kind of pumps as may be desired. The term liquid pump refers to a pump that may often pump liquid, but it is also possible that at certain times, such as when such a pump is operated in its reverse direction, such a pump may pump gas.

The term pressure measuring device is intended to encompass a pressure transducer, a pressure transmitter, and any other suitable device for measuring pressure.

In general, for harvesting cells, any combination or sequence of vibration or flow patterns or exposure to detachment reagent may be used. Detachment reagent may include an enzyme such as trypsin or collagenase or others.

In general, any combination of disclosed features, components and methods described herein is possible. Steps of a method can be performed in any order that is physically possible.

All cited references are incorporated by reference herein.

Although embodiments have been disclosed, it is not desired to be limited thereby. Rather, the scope should be determined only by the appended claims.

We claim:

1. A bioreactor system for culturing cells,
said bioreactor system comprising spatially fixed scaffolds upon which said cells can grow,
said bioreactor system having a liquid supply system for perfusing liquid through said scaffolds,
wherein said bioreactor system comprises a plurality of culture chambers each containing some of said scaffolds, said culture chambers having respective flow paths therethrough for flow of said liquid,
wherein said bioreactor system comprises a plurality of reservoirs or a plurality of sub-reservoirs,
wherein said bioreactor system has a control device to direct, to various of said plurality of culture chambers at a given time, respective flows of said liquid that are different from flows to others of said culture chambers with respect to flowrate of said liquid or flow direction of said liquid or duration of flow of said liquid.

2. The bioreactor system of claim 1, wherein said control device comprises at least one sensing device selected from the group consisting of a pH sensor, a dissolved oxygen sensor, a glucose sensor, a lactate sensor, a camera, and a device indicating a flow resistance of one of said scaffolds, wherein said control device is responsive to said at least one sensing device.

3. The bioreactor system of claim 1, wherein more than one of said plurality of said culture chambers are associated with a common reservoir of said liquid.

4. The bioreactor system of claim 1, wherein said culture chambers are each associated with a respective sub-reservoir, wherein each sub-reservoir isolates liquid contained therein from liquid in any other sub-reservoir.

5. The bioreactor system of claim 1, wherein at least some of said culture chambers are each associated with a different sub-reservoir, wherein each sub-reservoir isolates liquid contained therein from liquid in any other sub-reservoir, wherein some of said culture chambers are in fluid communication with others of said culture chambers by a flowpath through a side-flow filter located at an elevation above a liquid level in said sub-reservoir.

6. The bioreactor system of claim 1, wherein said control device comprises a plurality of pumps, and wherein each of said pumps connected so as to pump said liquid through only one of said culture chambers or a subset of said plurality of said culture chambers,

7. The bioreactor system of claim 1, wherein said control device comprises valves that can adjust distribution of flow of said liquid among said plurality of said culture chambers.

8. The bioreactor system of claim 1, wherein said liquid is one of a culture medium, a harvesting reagent and a saline solution.

9. The bioreactor system of claim 1, wherein a time for initiating harvesting of cells in one culture chamber is different from a time for initiating harvesting of cells in another culture chamber.

10. The bioreactor system of claim 1, wherein a time for initiating harvesting of cells in a particular culture chamber is responsive to a parameter measured for a culture medium in a particular culture chamber, said parameter being selected from the group consisting of: pH of said culture medium; dissolved oxygen concentration in said culture medium; glucose concentration in said culture medium; lactate concentration in said culture medium; electrical capacitive properties of said culture medium; an optical image of one of said scaffolds; and a flow resistance of one of said scaffolds.

11. The bioreactor system of claim 1, wherein said bioreactor system has a control device to direct, to various of said plurality of culture chambers at a given time, said respective flows of said liquid so as to create a liquid-gas interface in a first one of said culture chambers so as to have a liquid-gas interface elevation that is different from a liquid-gas interface elevation of a liquid-gas interface in another one of said culture chambers.

12. A method for retrieving cells from a bioreactor system, the method comprising:
providing a bioreactor system comprising a spatially fixed scaffold upon which said cells can grow, said bioreactor system having a liquid supply system for perfusing a liquid through said scaffolds, wherein said bioreactor system comprises a culture chamber containing some of said scaffolds, said culture chamber having a flow path therethrough for flow of said liquid;
culturing cells in said bioreactor on said scaffold; and
performing, in any combination and in any sequence, any one or more of:
exposing said cells to a harvesting reagent;
applying vibration to said bioreactor system;
applying oscillatory flow of liquid through said scaffold;
applying pulsatile flow of liquid through said scaffold; or
causing a liquid-gas interface to pass through said scaffold.

13. The method of claim 12, wherein said oscillatory flow or said passage of said gas-liquid interface has a flow frequency and said vibration has a vibration frequency, and one of said frequencies is identical to or is an integer multiple of the other of said frequencies.

14. The method of claim 13, wherein said vibration and said flow or said passage of said interface are applied in a phase relationship so as to reinforce each other.

15. The method of claim 12, wherein, in at least one of said culture chambers, said control device causes said liquid-gas interface to pass from a lowest of said scaffolds to an uppermost of said scaffolds.

16. The method of claim 12, wherein, in at least one of said culture chambers, said control device causes a flow direction of said liquid to change direction.

17. The method of claim 12, wherein said harvesting liquid comprises a triblock copolymer or a surfactant.

18. A method of culturing cells, said method comprising:
providing a bioreactor system comprising a spatially fixed scaffold upon which said cells can grow, said bioreactor system having a liquid supply system for perfusing a liquid through said scaffolds, said liquid supply system comprising a pump, wherein said liquid supply system comprises a pressure measuring device for measuring a pressure generated by said pump or a means for measuring electrical power consumed in operating said pump;
culturing cells on said scaffolds;
optionally harvesting said cells that have been cultured; and
during either said culturing or said harvesting or both, determining a flow resistance of said scaffold using information about flowrate of said liquid in combination with either information about said pressure measured by said pressure measuring device or information about said electrical power consumption of said pump.

19. The method of claim 18, further comprising using said flow resistance to adjust a process parameter or a duration of said culturing of said cells.

20. The method of claim 18, further comprising using said flow resistance to adjust a process parameter or a duration of said harvesting of said cells.
